(19) United States
(12) Patent Application Publication
(10) Pub. No.: US 2012/0108554 A1
(43) Pub. Date: May 3, 2012

(54) PROCESS FOR PREPARING HIGH PURITY 1alpha-HYDROXY VITAMIN D2

(75) Inventors: Bang-I Liou, Taoyuan (TW); Ching-Peng Wei, Taoyuan (TW)

(73) Assignee: FORMOSA LABORATORIES, INC., Taoyuan (TW)

(21) Appl. No.: 12/914,739

(22) Filed: Oct. 28, 2010

(51) Int. Cl. A61K 31/592; A61P 3/02; C07C 401/00

(52) U.S. Cl. 514/167; 552/653

(57) ABSTRACT

The present invention relates to a process for purifying Doxercalciferol, a synthetic vitamin D analog, also known as 1α-hydroxy vitamin D₂, to the purity greater than 99.5% by crystallization from a mixed solvent of methanol and acetonitrile. Each of the individual impurities can be controlled no more than 0.1% which meets the individual unknown impurities specification requirement of International Conference on Harmonisation (ICH) guideline. The crystallization yield is more than 75% which is suitable for employed as a commercial process.

### FIELD OF THE INVENTION

The present invention relates to a process for preparing 1α-hydroxy vitamin D₂, and more particularly to methods for its purification.

### BACKGROUND OF THE INVENTION

Doxercalciferol, also known as 1α-hydroxy vitamin D₂, is a synthetic vitamin D₂ analog that undergoes metabolic activation in vivo to form 1,25-dihydroxyvitamin D₂ (1,25-(OH)₂D₂), a naturally occurring, biologically active form of vitamin D₂. Doxercalciferol is a colorless crystalline compound with a calculated molecular weight of 412.66 and a molecular formula of C₂₈H₄₄O₂. It is soluble in oils and organic solvents, but is relatively insoluble in water. Chemically, doxercalciferol is (1α,3β,5Z,7E,22E)-9,10-secoergosta-5,7,10(19),22-tetraene-1,3-diol.

Many methods of 1α-hydroxylation have been reported. Some of these methods use steroid starting materials that are first hydroxylated and then converted to the corresponding vitamin D compound. See, e.g., U.S. Pat. No. 4,670,190 issued to Hesse; U.S. Pat. No. 4,022,891 issued to Takeshita; U.S. Pat. No. 3,966,77 issued to Mazur; U.S. Pat. No. 3,907,843 issued to DeLuca et al. Others directly hydroxylate vitamin D compounds. See, e.g., U.S. Pat. No. 4,338,250 issued to DeLuca et al.; U.S. Pat. No. 4,202,829 issued to DeLuca et al.; U.S. Pat. No. 4,263,215 issued to Hesse et al.; U.S. Pat. No. 4,772,433 issued to Hesse; U.S. Pat. No. 4,554,105 issued to Hesse. Some proceed via a cyclovitamin. See, e.g., U.S. Pat. No. 4,555,364 issued to DeLuca et al.; U.S. Pat. No. 4,260,549 issued to DeLuca et al.; U.S. Pat. No. 4,195,027 issued to DeLuca et al. Still others provide a total synthesis of the desired vitamin D compound from simple precursors. Most produce poor yields of desired product. Each new synthesis claims to simplify those which came before. However, even those that claim to be simpler, more efficient methods still require considerable separately steps, as, for example, by chromatography, in the synthesis.

Despite recognition of the need for a simple, straight forward method for producing 1α-hydroxy vitamin D₂, the art has not yet responded with such a method for the preparation of high purity 1α-hydroxy vitamin D₂.

There is therefore a need for a process which will enable 1α-hydroxy vitamin D₂ to be prepared more simply and/or in higher purity than hitherto possible, thus providing a process which would be sufficiently economic for commercial production.

### SUMMARY OF THE INVENTION

The present invention is related to a process for preparing a high purity compound having formula I

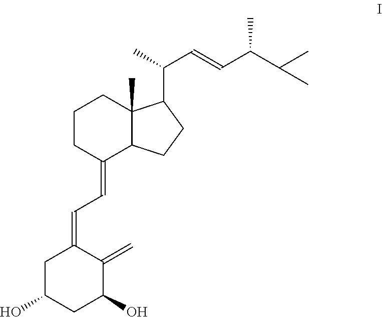

comprising the steps of:
(a) dissolving the crude compound in an alcoholic solvent;
(b) adding acetonitrile into the solution of step (a); and
(c) cooling the solution of step (b), followed by a filtration to obtain the high purity product.

In one aspect, the present invention provides a process to produce 1α-hydroxy vitamin D₂ of the purity greater than 99.5% and each of the individual impurities is no more than 0.1% as shown in FIG. 1.

In another aspect, the present invention also provides a crystalline form of 1α-vitamin D₂ having an X-ray diffraction pattern substantially as depicted in FIG. 2 and a DSC spectrum as shown in FIG. 3.

In another aspect, the present invention provides a pharmaceutical composition comprising the crystalline form of the present invention.

In further aspect, the present invention provides a method of preparing a pharmaceutical composition of the present invention comprising mixing the crystalline form of the present invention with a pharmaceutically acceptable carrier.

### BRIEF DESCRIPTION OF THE DRAWINGS

FIG. 1 illustrates ultra performance liquid chromatography (UPLC) for the purity.

FIG. 2 illustrates X-ray diffraction (XRD) of the crystalline form.

FIG. 3 illustrates differential scanning calorimetry (DSC) of the crystalline form.

### DETAILED DESCRIPTION OF THE INVENTION

The crude Doxercalciferol is prepared by following the synthetic route as shown in Scheme 1.

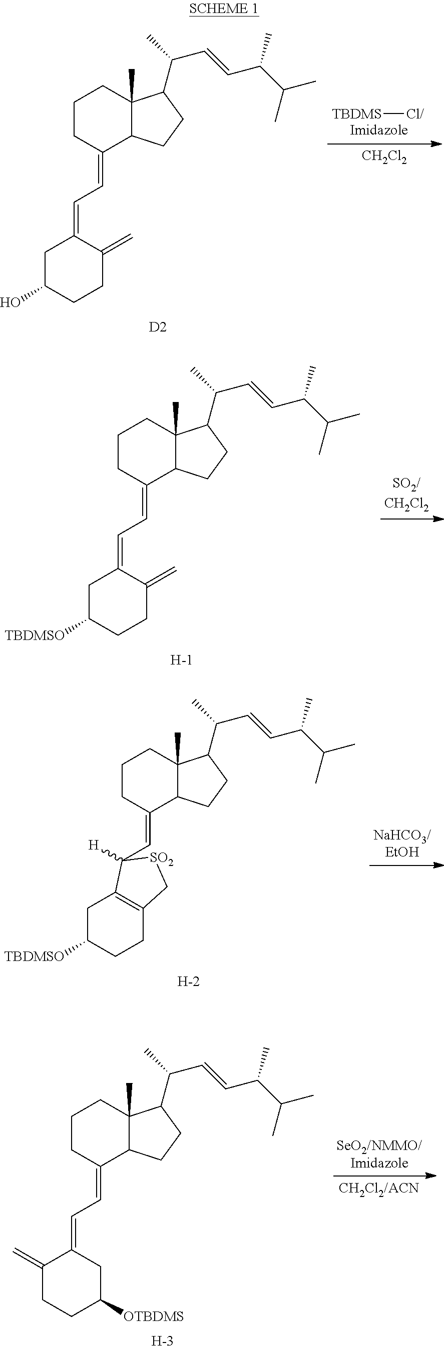

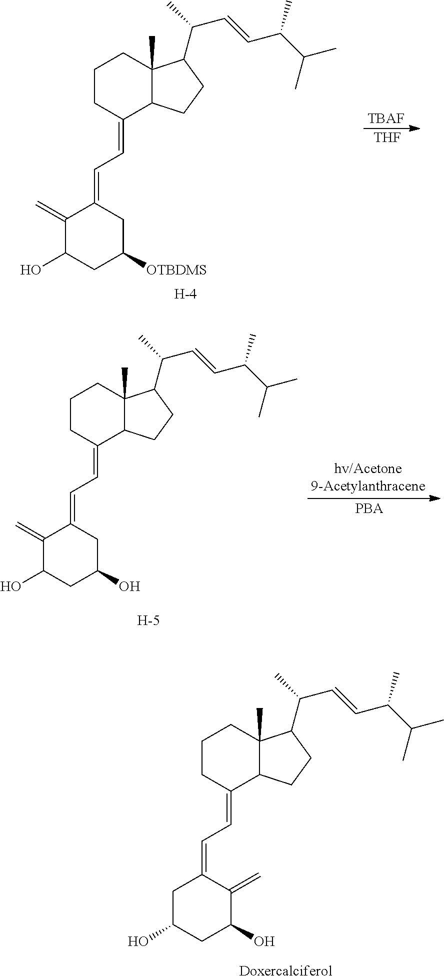

This preparation method is based on a combination of two patents, with some of our own modifications. U.S. Pat. No. 4,554,105 was followed for the preparation of the crude, and then U.S. Pat. No. 4,338,250 was followed to remove the 1β-isomer.

The present invention provides a process for preparing a high purity compound having formula I

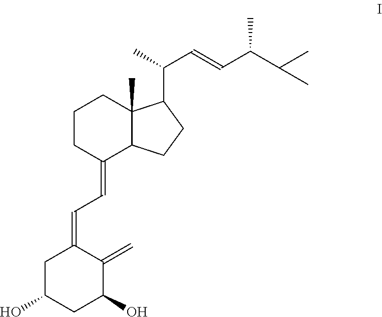

comprising the steps of:
(a) dissolving the crude compound in an alcoholic solvent;
(b) adding acetonitrile into the solution of step (a); and
(c) cooling the solution of step (b), followed by a filtration to obtain the high purity product.

In the process of the present invention, the alcohol solvent of step (a) is directed to a solvent to dissolve the compound of formula I. In one embodiment, the alcoholic solvent of step (a) is C1-C6 aliphatic alcohol, preferable methanol or ethanol, more preferable methanol.

To increase saturation of the solution, the present process can further comprises a step of concentration of the solution of step (a) between steps (a) and (b). In a preferred embodiment, the solution is concentrated to a weight of 5 to 10 times of the crude Doxercalciferol weight, more preferably 6 to 8 times.

In the present invention, acetonitrile is added into the said solution to increase the crystalline at step (b). The weight of acetonitrile added into the solution is 5 to 15 times of the crude Doxercalciferol weight, more preferably 7 to 12 times.

In the present invention, the cooling process at step (c) is performed at −15 to 35° C., preferably −5 to 20° C., more preferably 0 to 10° C.

In the present invention, steps (a)-(c) can be repeated to increase the purity of the final compound.

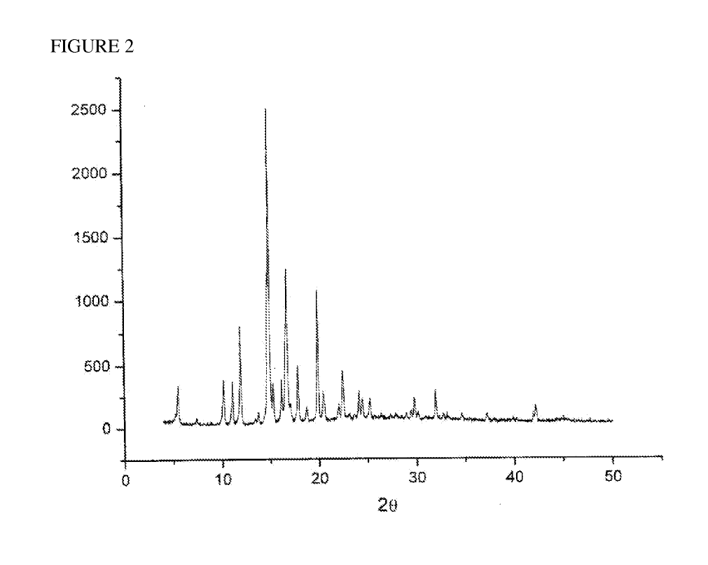

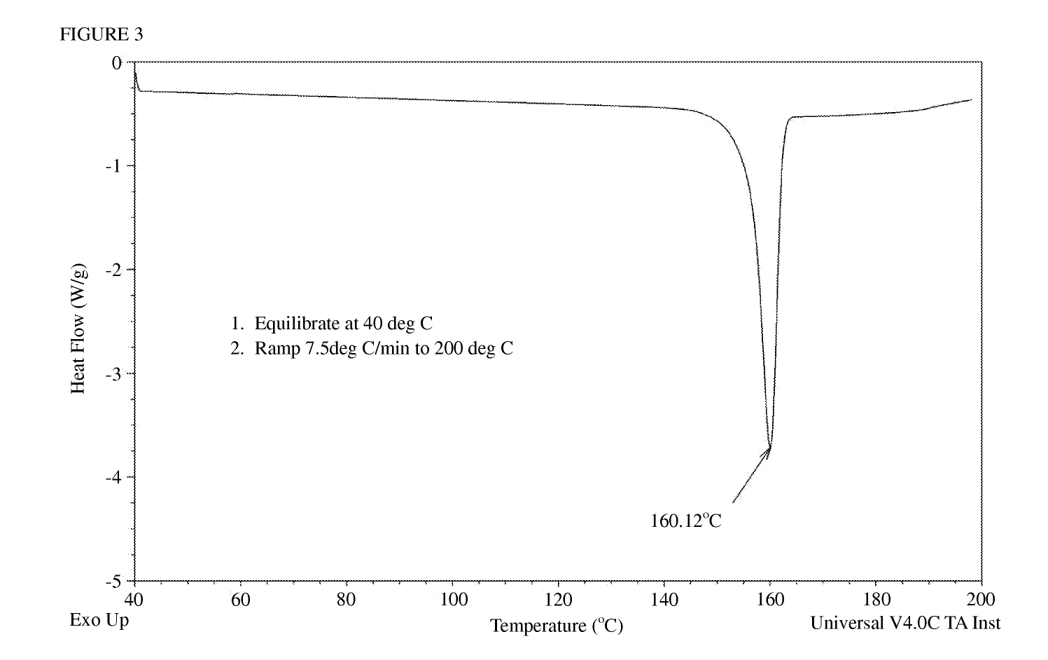

The present invention also provides a crystal form having an X-ray diffraction pattern substantially as depicted in FIG. 2 and a DSC spectrum as shown in FIG. 3.

Accordingly, the present invention provides a pharmaceutical composition comprising the crystalline form of the present invention.

The present invention further provides a method of preparing a pharmaceutical composition of the present invention comprising mixing the crystalline form of the present invention with a pharmaceutically acceptable carrier.

### EXAMPLE

The examples below are non-limited and are merely representative of various aspects and features of the present invention.

### Example 1

### Doxercalciferol Purification Procedure

19.9 g Crude Doxercalciferol and 378 g methanol (MeOH) were added into a 1 L 1-neck flask, and the mixture was stirred until the solid was completely dissolved. The solution was filtered and concentrated to a weight of 138 g. To the concentrated solution, 175 g acetonitrile was added and cooled to 0 to 10° C. with an ice bath for about 3 hours to obtain a suspension. The suspension was filtered and dried to obtain 16.9 g1st purified Doxercalciferol. The 1st purified Doxercalciferol and 321 g MeOH were added into a 1 L 1-neck flask, and the mixture was stirred until the solid was completely dissolved. The solution was filtered and concentrated to a weight of 117 g. The concentrated solution was added 149 g acetonitrile and cooled to 0 to 10° C. with an ice bath for about 3 hours to obtain a suspension. The suspension was filtered and dried to obtain 13.3 g 2nd purified Doxercalciferol with 99.89% purity and the individual impurity contents are less than 0.10%, as determined by UPLC.

### Example 2

### UPLC Analysis for the Purity

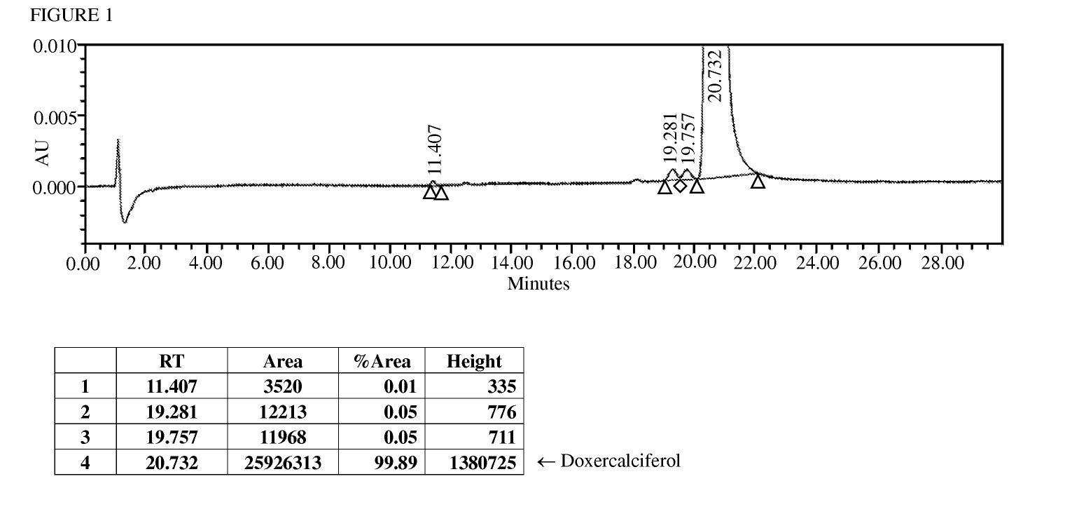

The purity analysis was performed with a Waters Acquity™ Ultra Performance LC (UPLC). The column system was two Waters Acquity UPLC® BEH C18, 2.1*100 mm, 1.7 μm column connected in series. The column temperature was 25° C. The mobile phase was Acetonitrile/H₂O=4/1(v/v). The flow rate was 0.4 mL/min. The UV wave length was 265 nm. The run time was 30 min. As illustrated in FIG. 1, the sample obtained in Example 1 showed a purity of 99.89% and the contents of the individual impurity was no more than 0.10%.

### Example 3

### X-Ray Diffraction and DSC Analysis for the Crystal Form

The sample obtained in Example 1 was analyzed with X-ray diffraction of the 2θ value as illustrated in FIG. 2. It indicated that the sample existed in a crystal form. The sample was also analyzed with differential scanning calorometry (DSC) of the condition: 1. equilibrate at 40° C., 2. ramp 7.5° C./min to 200° C. As illustrated in FIG. 3, there was a single melting point at 160.12° C. which indicated that this sample was a single crystal form.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A process for preparing a high purity compound having formula I 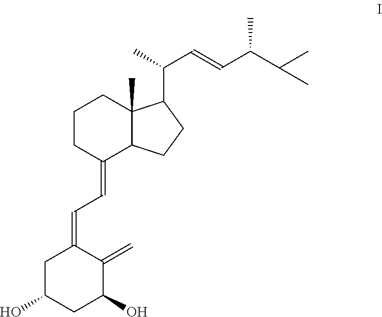
comprising the steps of:
(a) dissolving the crude compound in an alcoholic solvent;
(b) adding acetonitrile into the solution of step (a); and
(c) cooling the solution of step (b) followed by a filtration to obtain the high purity product.

2. The process according to claim 1, wherein the alcoholic solvent of step (a) is C1-C6 aliphatic alcohol.

3. The process according to claim 2, wherein the alcoholic solvent is methanol or ethanol.

4. The process according to claim 3, wherein the alcoholic solvent is methanol.

5. The process according to claim 1, which further comprises a step of concentration of the solution of step (a) between steps (a) and (b).

6. The process according to claim 5, wherein the solution of step (a) is concentrated to a weight of 5 to 10 times of the crude Doxercalciferol weight.

7. The process according to claim 6, wherein the solution of step (a) is concentrated to a weight of 6 to 8 times of the crude Doxercalciferol weight.

8. The process according to claim 1, wherein the weight of acetonitrile added into the solution is 5 to 15 times of the crude Doxercalciferol weight.

9. The process according to claim 8, wherein the weight of acetonitrile added into the solution is 7 to 12 times of the crude Doxercalciferol weight.

10. The process according to claim 1, wherein the cooling process is performed at −15 to 35° C.

11. The process according to claim 10, wherein the cooling process is performed at −5 to 20° C.

12. The process according to claim 11, wherein the cooling process is performed at 0 to 10° C.

13. The process according to claim 1, wherein the high purity is more than 99.5% and each of the individual impurity contents is no more than 0.1%.

14. The process according to claim 1, which further comprises repeating steps (a) to (c) to increase the purity of the final compound.

15. A crystal form having an X-ray diffraction pattern substantially as depicted in FIG. 2 and a DSC spectrum as shown in FIG. 3.

16. A pharmaceutical composition comprising the crystal form according to claim 1.

17. A method of preparing a pharmaceutical composition of claim 1 comprising mixing the crystal form according to claim 1 with a pharmaceutically acceptable carrier.
